(19) United States
(12) Patent Application Publication
(10) Pub. No.: US 2026/0176309 A1
(43) Pub. Date: Jun. 25, 2026

(54) ENDOLYSIN WITH ENHANCED ACTIVITY AGAINST BIOFILM PNEUMOCOCCI AND NASOPHARYNGEAL COLONIZATION AND METHODS THEREOF

(71) Applicant: University of Maryland, College Park, College Park, MD (US)

(72) Inventors: Daniel C. NELSON, Rockville, MD (US); Adit B. ALREJA, Silver Spring, MD (US); Sara Beth LINDEN, Bethesda, MD (US)

(73) Assignee: University of Maryland, College Park, College Park, MD (US)

(21) Appl. No.: 19/065,347

(22) Filed: Feb. 27, 2025

Related U.S. Application Data
(60) Provisional application No. 63/558,402, filed on Feb. 27, 2024.

(51) Int. Cl. C07K 14/315; A61K 9/00; A61K 38/00; A61K 45/06; A61P 31/04

(52) U.S. Cl. CPC C07K 14/3156; A61K 9/0043; A61K 45/06; A61P 31/04; A61K 38/00; C07K 2319/02

(57) ABSTRACT

The present disclosure is drawn to an endolysin protein against Streptococcus pneumoniae (Spn), designated SP-CHAP. The disclosure provides methods of treating diseases in a subject resulting from infection with pneumococcal bacteria, said diseases including but not limited to pneumonia, invasive pneumococcal disease, meningitis and sepsis. Such treatment methods include administering to the subject a therapeutically effective amount of a pharmaceutical composition comprising SP-CHAP polypeptide, polypeptide fragments, SP-CHAP encoding nucleic acids (RNA/DNA) or variants thereof.

This application claims priority to provisional application Ser. No. 63/558,402, filed Feb. 27, 2024, the entire contents of which are incorporated herein.

### GOVERNMENT SUPPORT STATEMENT

This invention was made with government support under R01 AI16813 awarded by the National Institutes of Health. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Feb. 3, 2026, is named “1475-127.xml” and is 3,966 bytes in size. The sequence listing contained in this .XML file is part of the specification and is hereby incorporated by reference herein in its entirety.

### TECHNICAL FIELD

The present disclosure is drawn to an endolysin protein against Streptococcus pneumoniae (Spn), designated SP-CHAP. The disclosure provides methods of treating diseases in a subject resulting from infection with pneumococcal bacteria, said diseases including but not limited to pneumonia, invasive pneumococcal disease, meningitis and sepsis. Such treatment methods include administering to the subject a therapeutically effective amount of a pharmaceutical composition comprising SP-CHAP polypeptide, polypeptide fragments, SP-CHAP encoding nucleic acids (RNA/DNA) or variants thereof.

### BACKGROUND

Endolysins offer an alternative to conventional antibiotics in addressing the emergence of multi-drug-resistant bacteria. These enzymes, encoded by bacteriophages, induce cell lysis upon synthesis at the end of the replication cycle and are not susceptible to efflux pumps, penicillin-binding proteins, or other common mechanisms of antimicrobial resistance (1, 2). Moreover, development of any resistance to endolysins has not been reported (3).

Streptococcus pneumoniae (Spn), a Gram-positive nasopharyngeal pathobiont, can cause infections including pneumonia, invasive pneumococcal disease, meningitis, and sepsis (4, 5). As novel antibiotic approval rates have declined and frequency of antibiotic-resistant strains of Spn have surged, addressing this pathogen is imperative (6). Several endolysins that target Spn, such as Cpl-1 (7) and Pal (8), have shown efficacy both in vitro and in vivo. Many endolysins share a common cysteine, histidine-dependent amidohydrolase/peptidase (CHAP) domain (9). Several CHAP-containing endolysins with antimicrobial activity against Staphylococcus aureus, including N-Rephasin® (SAL200) and Exebacase (CF-301), have been investigated in human clinical trials (3). However, no naturally occurring pneumococcal endolysin with a CHAP domain has been described.

In light of the high rates of pneumococcal resistance reported for several antibiotics, alternatives are urgently needed. Provided herein is an endolysin targeting Spn that is more active than Cpl-1, the most characterized pneumococcal endolysin to date, and which provides an alternative to pneumococcal bacterial infection.

### SUMMARY

It has been discovered that SP-CHAP, a newly identified Spn endolysin, possesses the ability to lyse Spn and decolonize biofilm-grown bacteria both in vitro and in vivo. Accordingly, the present disclosure provides compositions and methods for treatment of a subject suffering from a pneumococcal infection, or at risk of developing a pneumococcal infection, comprising administering to the subject, an effective amount of SP-CHAP in a pharmaceutically acceptable form.

In an embodiment, pharmaceutical compositions comprising SP-CHAP and a pharmaceutical acceptable carrier are provided. The SP-CHAP exhibits properties for use as therapeutic agents, e.g., in the treatment of pneumococcal infection-based diseases. In addition, certain embodiments relate to pharmaceutical compositions comprising polynucleotides encoding SP-CHAPs, vectors, and host cells comprising such SP-CHAPs. The protein sequence of SP-CHAP is listed in SEQ ID NO:1, while an optimized SP-CHAP encoding nucleic acid is listed in SEQ ID NO:2.

In one aspect of the present disclosure provides an anti-bacterial, anti-pneumococcal composition comprising SP-CHAP protein or nucleic acids encoding SP-CHAP (herein referred to collectively as “SP-CHAP reagents”), as an active ingredient. The compositions disclosed herein refer to a composition able to prevent infection or re-infection with a bacterial pathogen. In a non-limiting embodiment, the bacteria is pneumococcal bacteria and the antibacterial composition is able to reduce the severity of symptoms or eliminate the symptoms of bacterial infection, or substantially or completely remove the symptoms of said infection. Said infection, may be in the nasal cavity, but may also spread to the other parts of the body such as the lung, the eustachian tube and ear.

The anti-bacterial compositions provided herein may be prepared in any suitable and pharmaceutically acceptable formulation. It may be provided in the form of an immediately administrable solution or suspension, or a concentrated crude solution suitable for dilution before administration or may be provided in a form capable of being reconstituted, such as a lyophilized, freeze-dried, or frozen formulation. In a specific embodiment, the anti-bacterial composition is formulated for intranasal administration.

The anti-bacterial composition may contain a pharmaceutically acceptable carrier in order to be formulated. The carrier typically includes a diluent, an excipient, a stabilizer, a preservative, and the like. In a specific embodiment of the invention, the anti-bacterial composition is formulated for intranasal administration. In another embodiment, the anti-bacterial composition is formulated for systemic administration, for example, intramuscular administration.

In another aspect, compositions comprising nanoparticles or liposomes and the disclosed SP-CHAP reagents. Nanoparticles or liposomes can be created from biological molecules or from non-biological molecules. In some cases, the SP-CHAP reagents are crosslinked to a polymer or lipids on the nanoparticle or liposome surface. In embodiments, the SP-CHAP reagents are adsorbed onto the nanoparticle or liposome surface. In some embodiments, the disclosed SP-CHAP reagents are adsorbed onto the surface and then crosslinked to the surface. In some embodiments, disclosed SP-CHAP reagents are encapsulated into the nanoparticle or liposome. Such nanoparticles, or liposomes, may be incorporated into anti-bacterial compositions as disclosed below.

In one aspect, a mRNA-based approach may be utilized to deliver SP-CHAP to the site of infection. Such administration of SP-CHAP encoding nucleic acids (“mRNA delivery system”) would enable endogenous synthesis and production of the endolysin within target organs or tissues. Contact of SP-CHAP RNA to cells, such as human cells, would result in the expression and production of SP-CHAP in the cytosol of the contacted cell.

In still another aspect, a method is provided for preparation of the SP-CHAP provided herein. The preparation methods according to the present disclosure may be performed through recombinant DNA methods.

A method of treating a subject is provided that includes administering a disclosed anti-bacterial composition comprising SP-CHAP reagents, as described herein, to a subject in need thereof. In a non-limiting embodiment, the bacteria is a pneumococcal bacterium, and the composition is able to reduce the severity of symptoms or eliminate the symptoms of the bacteria infection, or substantially or completely remove the disease caused by the bacteria infection. The compositions disclosed herein may also be administered prophylactically to a subject, e.g., a human, before infection with the bacteria, or may be therapeutically administered to subjects after infection with the bacteria.

The present disclosure provides a kit that includes the SP-CHAP compositions, as described herein. In one specific aspect the kit further includes instructions for the treatment and/or prophylaxis of the pneumococcal infection. The anti-bacterial compositions may, if desired, be presented in a pack or dispenser device which may contain one or more-unit dosage forms containing the SP-CHAP composition. In a specific embodiment, the dispenser may be one to be used for intranasal administration of the composition. In a specific embodiment, the dispenser may be one to be used for intramuscular administration of the composition. The pack may for example include metal or plastic foil, such as a blister pack. The pack or dispenser device may be accompanied by instructions for administration to subjects, especially humans.

### BRIEF DESCRIPTION OF FIGURES

In order to better understand the subject matter that is disclosed herein and to exemplify how it may be carried out in practice, embodiments will now be described, by way of non-limiting example, with reference to the accompanying drawings. With specific reference to the drawings, it is stressed that the particulars shown are by way of example and for purposes of illustrative discussion of embodiments of the disclosure.

FIG. 1A-H. Endolysin SP-CHAP is highly active against planktonic-grown pneumococci and its activity is modulated by capsule polysaccharide availability. Biochemical characterization of optimal conditions for SP-CHAP activity. The role of pH (FIG. 1A), temperature (FIG. 1B), and salt (FIG. 1C) on SP-CHAP lytic activity against Spn DCC1811. Lytic activity of 50 μg/mL Cpl-1 and SP-CHAP on (FIG. 1D) Spn strains TIGR4, D39, DCC1811, 1335, and Lyt4.4 or (FIG. 1E) the commensal staphylococcal bacteria (S. hominis and S. epidermidis) and commensal streptococcal bacteria (S. gordonii, S. intermedius, S. mitis, and S. salivarius) as measured by OD600. (FIG. 1F) Lytic activity of SP-CHAP and Cpl-1 at various concentrations on Spn Lyt4.4 as measured by the CFU assay. Data are reported as the log-fold killing compared to untreated controls. (FIG. 1G) Minimum inhibitory action MIC (μg/mL of endolysin) of SP-CHAP and Cpl-1 on Spn strains TIGR4, DCC1811, DCC1335, Lyt4.4, D39, and R6. (H) Lytic activity of 50 μg/mL Cpl-1 and SP-CHAP on Spn strain Lyt4.4 after incubation with purified pneumococcal capsular polysaccharide. Experiments were done in triplicate, and the error bars represent the standard deviations. For two-way ANOVA with Dunnett's multiple comparisons test, asterisks denote the level of significance observed: ns=not significant; *=p≤0.05, **=p≤0.01, ***=p≤0.001, ***=p≤0.0001.

FIG. 2A-G. SP-CHAP reduces pneumococcal biofilm biomass in vitro and nasopharyngeal colonization in vivo. (FIG. 2A) Disruption of Spn Lyt4.4 biofilms by PBS, or 1.56 μg/mL Cpl-1 or SP-CHAP, as visualized by crystal violet staining. (FIG. 2B) Quantification of biomass measured by OD595 of crystal violet stained biofilms. SP-CHAP displayed biofilm eradication ability at all concentrations tested while Cpl-1 removed biofilms at concentrations as low as 12.5 μg/mL. Statistical analysis denotes differences between PBS and endolysin-treated wells. (FIG. 2C) 630× confocal microscopy consisting of nineteen 0.5 μm slices of Spn biofilms treated with PBS, Cpl-1, or SP-CHAP. Scale bar=10 μm. (FIG. 2D) Viability of Spn Lyt4.4 biofilms after treatment with 20 μg/mL Cpl-1 or SP-CHAP for one hr as measured by the CFU assay. Data are reported as the Log CFU/mL of recovered bacteria. (FIG. 2E) Acute systemic toxicity in mice intraperitoneally challenged with vehicle or SP-CHAP. (FIG. 2F) Sketch of mouse colonization model and (FIG. 2G) Log CFU per gram of nasopharyngeal tissue from mice infected with Spn strain TIGR4 and treated with 60 μg of endolysin 48 hr post-colonization and samples collected 4 hr post-treatment. Samples tested with two-way ANOVA or one-way ANOVA Kruskal-Walls test with Dunn's multiple-comparison post-test. Asterisks denote the level of significance observed: ns=not significant; *=p≤0.05, **=p≤0.01, ***=p≤0.001, ****=p≤0.0001.

### DETAILED DESCRIPTION

### Definitions

The term “pneumococcal disease” as used herein means a condition or disease characterized by infection with a pneumococcal bacteria. In one embodiment, the pneumococcal bacteria is Streptococcus pneumoniae (Spn)

The term “pneumococcal-based disease” as used herein means a condition or disease characterized by a pneumococcal bacteria infection in a subject. Such diseases include, for example, those resulting from nasal colonization, middle ear infection, otitis media, pneumonia, invasive pneumococcal disease, meningitis and sepsis. In such diseases SP-CHAP may be administered to mediate lysis of the infecting bacteria.

The terms “effective amount” or “therapeutically effective amount” as used herein have the standard meanings known in the art and are used interchangeably herein to mean an amount sufficient to treat a subject afflicted a pneumococcal-based disease, or to halt the progression of the condition or disease or alleviate a symptom or a complication associated with the disease. The exact dose will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999)).

The terms “protein” and “polypeptide” as used herein are used interchangeably, unless specified to the contrary, and according to conventional meaning, mean a sequence of amino acids. Peptides are not limited to a specific length, e.g., they may comprise a full-length protein sequence or a fragment of a full-length protein. Said proteins may include variants and may include variants affecting (for example, inhibiting) post-translational modifications of the polypeptide, for example, glycosylations, acetylations, phosphorylations and the like, as well as other modifications known in the art, both naturally occurring and non-naturally occurring, e.g., variants.

The term “subject” as used herein refers to an animal. Typically, the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human. A subject in need is a subject that is suffering from a pneumococcal infection.

The term “therapeutic agent” as used herein is a compound capable of producing a desired and beneficial effect.

The terms “treat,” “treating” or “treatment” of any disease or disorder as used herein refer in one embodiment, to halting the progression of the condition or disease, or to ameliorating the disease or disorder (i.e., slowing or arresting or reducing the development of the disease or at least one of the clinical symptoms thereof). In another embodiment “treat,” “treating” or “treatment” refers to alleviating or ameliorating at least one physical parameter including those which may not be discernible by the patient. In yet another embodiment, “treat,” “treating” or “treatment” refers to modulating the disease or disorder, either physically, (e.g., stabilization of a discernible symptom), physiologically, (e.g., stabilization of a physical parameter), or both. In yet another embodiment, “treat,” “treating” or “treatment” refers to preventing or delaying the onset or development or progression of the disease or disorder. As used herein, a subject is “in need of” a treatment if such subject would benefit biologically, medically or in quality of life from such treatment.

The term “vector” as used herein refers to any molecule or entity (e.g., nucleic acid, plasmid, bacteriophage or virus) used to transfer protein coding information into a host cell. Certain vectors are capable of directing the expression of nucleic acids to which they are operatively linked. Such vectors are referred to herein as “expression vectors.” An “expression vector” or “expression construct” as used herein refers to a recombinant DNA molecule containing a desired coding sequence and appropriate nucleic acid control sequences necessary for the expression of the operably linked coding sequence in a particular host cell.

As used herein, the term “SP-CHAP” refers to a protein having the following amino acid sequence (SEQ ID NO:1):

MSKKQEMIQFFIDKANSGDGVDNDGAYGFQCADVPCYGLRHWYGVTLWGN
AYDLLESARSQGLKVVYDAEYPKAGWFFVKSYVAGDGVNYGHTGLVYEDS
DGSTIKTIEQNIDGNWDYLEVGGPCRYNERSVSEIVGYIVPPEEVEIGWQ
QNQYGWWWVREDGSYPTEKWEKINDVWYYFDDKGFMKRSTWLNYKDAWYW
FTDSGSMAIGWARINNAWYYFDEEGKMVTGWIKHKLTWYYLDGKEGTMVS
NAFIQSADKKGWYYLKPDGTMEDKPEFEIEPDGLITTK

Such SP-CHAPs, also include polypeptide fragments of SP-CHAP, as well as variants of the protein. Full length protein, polypeptide fragments and variants are collectively referred to herein in “SP-CHAPs”.

In a non-limiting embodiment, a SP-CHAP encoding nucleic acid (SEQ ID NO:2) includes the following:

ATGAGCAAAAAGCAAGAGATGATCCAGTTCTTCATCGATAAAGCAAATAG
CGGTGATGGCGTTGATAATGATGGTGCATATGGTTTTCAGTGTGCAGATG
TTCCGTGTTATGGTCTGCGTCATTGGTATGGTGTTACCCTGTGGGGTAAT
GCATATGACCTGCTGGAAAGCGCACGTAGCCAGGGTCTGAAAGTTGTTTA
TGATGCAGAATATCCGAAAGCCGGTTGGTTTTTCGTTAAAAGCTATGTTG
CCGGTGATGGTGTGAATTATGGTCATACCGGTCTGGTTTATGAAGATAGT
GATGGTAGCACCATTAAAACCATCGAACAGAATATTGATGGCAACTGGGA
TTATCTGGAAGTTGGTGGTCCGTGTCGTTATAATGAACGTAGCGTTAGCG
AAATTGTGGGCTATATTGTTCCGCCTGAAGAAGTTGAAATTGGTTGGCAG
CAGAATCAGTATGGTTGGTGGTGGGTTCGTGAAGATGGTAGCTATCCGAC
CGAAAAATGGGAGAAAATTAACGATGTGTGGTACTACTTCGATGACAAGG
GTTTTATGAAACGTAGCACCTGGCTGAACTATAAAGATGCATGGTATTGG
TTTACCGATAGCGGTAGCATGGCCATTGGTTGGGCACGTATTAACAATGC
CTGGTATTATTTCGACGAAGAGGGTAAAATGGTTACCGGCTGGATTAAAC
ATAAACTGACGTGGTATTACCTGGATGGTAAAGAAGGCACCATGGTTAGC
AATGCATTTATTCAGAGCGCAGATAAGAAAGGCTGGTACTATCTGAAACC
GGATGGTACAATGGAAGATAAACCGGAATTTGAAATTGAGCCGGATGGTC
TGATTACCACCAAA

Such P-CHAP encoding nucleic acids include any nucleic acid encoding the SP-CHAP of SEQ ID NO: 1 as well as polypeptide fragments and variants of SP-CHAP.

In non-limiting embodiments, pharmaceutical compositions comprising SP-CHAP and a pharmaceutical acceptable carrier are provided. The SP-CHAP exhibits properties for use as a therapeutic agent, e.g., in the treatment of pneumococcal disease. In addition, certain embodiments relate to compositions comprising polynucleotides encoding SP-CHAP, vectors, and host cells comprising SP-CHAP.

In one aspect, the present disclosure provides methods for treating pneumococcal disease in a subject comprising administering to the subject, an effective amount of SP-CHAP reagents in a pharmaceutically acceptable form. The present disclosure further provides pharmaceutical compositions for treatment of pneumococcal disease to a subject in need.

In an embodiment, a method of treating a subject suffering from pneumococcal disease is provided, the method comprising administering to the subject, an effective amount of SP-CHAP reagents in a pharmaceutically acceptable form. Such pneumococcal disease include but are not limited to those resulting from nasal colonization, middle ear infection, otitis media, pneumonia, invasive pneumococcal disease, meningitis and sepsis.

For production of SP-CHAP, the polypeptides may be made in transformed host cells using recombinant DNA techniques. To do so, a recombinant DNA molecule coding for the SP-CHAP protein is prepared. Methods of preparing such DNA and/or RNA molecules are well known in the art. For instance, sequences coding for the SP-CHAP protein could be exercised from DNA using suitable restriction enzymes. The relevant sequences can be created using the polymerase chain reaction (PCR) with the inclusion of useful restriction sites for subsequent cloning. Alternatively, the DNA/RNA molecule could be synthesized using chemical synthesis techniques, such as the phosphoramidite method. Also, a combination of these techniques could be used.

Certain embodiments also include a vector encoding SP-CHAP in an appropriate host. The vector comprises the DNA molecule that encodes SP-CHAP operatively linked to appropriate expression control sequences. Methods of affecting this operative linking, either before or after the polypeptide-encoding DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation. The resulting vector comprising the protein-encoding DNA molecule is used to transform an appropriate host cell. This transformation may be performed using methods well known in the art.

Any of a large number of available and well-known host cells may be used in the practice of these embodiments. The selection of a particular host is dependent upon a number of factors recognized by the art. These factors include, for example, compatibility with the chosen expression vector, toxicity to the host cell of the proteins encoded by the DNA molecule, rate of transformation, ease of recovery of the proteins, expression characteristics, biosafety and costs. A balance of these factors must be struck with the understanding that not all hosts may be equally effective for the expression of a particular DNA sequence.

Next, the transformed host is cultured under conditions so that the desired SP-CHAP is expressed. Such conditions are well known in the art. Finally, the proteins are purified from the fermentation culture or from the host cells in which they are expressed. These purification methods are also well known in the art. SP-CHAP prepared as described herein may be purified by art-known techniques such as high-performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification, an antibody, ligand, receptor or antigen can be used to which the SP-CHAP binds. In addition, size exclusion chromatography can be used to isolate SP-CHAP.

In an embodiment, a method of producing a SP-CHAP is provided, wherein the method comprises culturing a host cell comprising a polynucleotide encoding the SP-CHAP under conditions suitable for expression of the SP-CHAP and recovering the SP-CHAP from the host cell (or host cell culture medium). The purity of the SP-CHAP can be determined by any of a variety of well-known analytical methods including gel electrophoresis, high pressure liquid chromatography, and the like.

The skilled artisan will readily appreciate that the embodiments are not limited to the SP-CHAP sequences depicted herein, but also includes variants of SP-CHAP. Such variants may contain deletions, substitutions or additions of one or more amino acids in the above depicted amino acid sequence of SEQ ID NO. 1 while maintaining the biological activity of naturally occurring SP-CHAP. Such variants include those, for example, that increase the half-life or stability of the SP-CHAP or increase the affinity and binding of SP-CHAP to the target pneumococcal bacterium. Such fragments or variants may be naturally occurring or may be synthetically generated, for example, by modifying one or more of the above peptide sequences used in the methods of certain embodiments and evaluating their effects using any of a number of techniques well known in the art.

Additionally, the bacterial SP-CHAP may be engineered to contain signal peptides so as to enable the secretion of the SP-CHAP from the expressing cell. Such signal peptides are short peptides located in expressed proteins, which carrying information for protein secretion. They are ubiquitous to all prokaryotes and eukaryotes. In non-limiting embodiments the signal peptide may be a bacterial signal peptide. In another embodiment, the signal peptide may be a eukaryotic signal peptide. In non-limiting embodiments, the signal peptide may be a lysozyme signal peptide, human immunoglobulin G heavy chain signal peptide or human azurocidin signal peptide.

In additional, non-limiting embodiments, the variant SP-CHAP is engineered to include a tag, label, targeting moiety or ligand, a cell binding motif or therapeutic agent, an antibacterial, or antibody. In an embodiment the SP-CHAP is engineered to transit the tympanic membrane for treatment of ear infection. The SP-CHAP may also be engineered to contain variants that inhibit undesirable post-translational modifications of the bacterial protein, e.g. glycosylation, when expressed in a mammalian cell.

Such SP-CHAP variants may include chimeric proteins, e.g., fusion SP-CHAP proteins. A “chimeric” polypeptide may be produced by combining two or more proteins having two or more functionally active sites. Chimeric polypeptides may act independently on the same or different molecules and hence may potentially exhibit activity against two or more different bacterial species or antigen targets. In an embodiment, the fusion of SP-CHAP to heterologous peptide or protein domains can impart additional desirable functionalities to the SP-CHAP such as targeting specific regions of the body, e.g., nasal cavity, the eustachian tubes, lung, etc.

As used herein, a peptide fragment or variant has amino acid sequences that are at least about 70-75%, typically at least about 80-85%, and most typically at least about 90-95%, 97%, 98% or 99% or more homologous with the SP-CHAP (SEQ ID NO. 1) or peptide fragments thereof. Also included, are nucleic acids encoding for the different variant polypeptides. In certain embodiments, a fragment or variant may contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides of certain embodiments and still obtain a functional molecule that encodes a variant or derivative polypeptide with desirable characteristics, e.g. lysin activity.

In SP-CHAP, suitable conservative substitutions of amino acids are known to those of skill in this art and generally can be made without altering the biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). One of skill in the art could determine which amino acid residues can be substituted, inserted, or deleted without abolishing biological activity. Assistance can be found using computer programs well known in the art, such as DNASTAR™ software. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

Fragments, or variants, or derivatives of SP-CHAP include aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and mutants. Truncations or deletions of regions which change functional activity of the proteins are also variants.

Methods of producing SP-CHAP, polypeptide fragments or variants thereof, for use in the methods disclosed herein may also be made using, for example, solid phase synthesis techniques may be used. Suitable techniques are well known in the art, and include those described in Merrifield, in Chem. Polypeptides, pp. 335-61 (Katsoyannis and Panayotis eds. 1973); Merrifield, J. Am. Chem. Soc. 85:2149 (1963); Davis et al., Biochem. Intl. 10:394-414 (1985); Stewart and Young, Solid Phase Peptide Synthesis (1969); U.S. Pat. No. 3,941,763; Finn et al., The Proteins, 3rd ed., vol. 2, pp. 105-253 (1976); and Erickson et al., The Proteins, 3rd ed., vol. 2, pp. 257-527 (1976).

Many vectors are known in the art for recombinant expression of polypeptides. Vector components may include one or more of the following: a signal sequence, an origin of replication, one or more selective marker genes (that may, for example, confer antibiotic or other drug resistance, complement auxotrophic deficiencies, or supply critical nutrients not available in the media), an enhancer element, a promoter, and a transcription termination sequence, all of which are well known in the art.

An “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription and translation.

Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. Such techniques are well known in the art. (E.g., Goodey, Andrew R.; et al., Peptide and DNA sequences, U.S. Pat. No. 5,302,697; Weiner et al., Compositions and methods for protein secretion, U.S. Pat. Nos. 6,022,952 and 6,335,178; Uemura et al., Protein expression vector and utilization thereof, U.S. Pat. No. 7,029,909; Ruben et al., 27 human secreted proteins, US 2003/0104400 A1), the contents of which are hereby incorporated by reference.

A “secreted” protein refers to those proteins capable of being directed to the ER, secretory vesicles, or the extracellular space as a result of a secretory signal peptide sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

In one embodiment, a vector, preferably an expression vector, comprising one or more of the polynucleotides encoding the SP-CHAP of certain embodiments is provided. In some embodiments, the vector is introduced into mammalian cells, e.g., HEK293 or CHO cells to produce the SP-CHAP in supernatant for purification. The resulting SP-CHAP can then be used in pharmaceutical compositions for use in treatment of pneumococcal diseases.

Methods are well known to one of skill in the art and can be used to construct expression vectors containing the coding sequence with appropriate transcriptional and translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989).

Typically, the expression vectors are derived from virus, plasmid, prokaryotic or eukaryotic chromosomal elements, or some combination thereof, and may optionally include at least one origin of replication, at least one site for insertion of heterologous nucleic acid, and at least one selectable marker. An expression vector may be any suitable DNA or RNA vector, including chromosomal, non-chromosomal, and synthetic nucleic acid vectors (a nucleic acid sequence comprising a suitable set of expression control elements). Examples of such vectors include derivatives of SV40 bacterial plasmids, phage DNA, baculovirus, yeast plasmids, vectors derived from combinations of plasmids and phage DNA, and viral nucleic acid (RNA or DNA) vectors. Those skilled in the art will be able to readily select the proper vectors, expression control sequences, and hosts to achieve the desired expression.

In such vectors, typically, a promoter region would be operably associated with a nucleic acid encoding SP-CHAP if the promoter was capable of effecting transcription of that nucleic acid. The promoter can be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are known to those skilled in the art.

A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit a-globin, as well as other sequences capable of controlling gene expression in eukaryotic cells. Additional suitable transcription control regions include tissue-specific promoters and enhancers as well as inducible promoters.

The term “host cell” means a cell that has been transformed, or is capable of being transformed, with a nucleic acid and thereby expresses a gene of interest. The polynucleotides encoding the SP-CHAP for therapeutic use may be expressed in any appropriate host cell. The host cell can be prokaryotic (bacteria) or eukaryotic (e.g., yeast, insect, plant and animal cells).

Exemplary mammalian host cells are COS1 and COS7 cells, NSO cells, Chinese hamster ovary (CHO) cells, NIH 3T3 cells, HEK293 cells, HEPG2 cells, HeLa cells, L cells, MDCK, W138, murine ES cell lines (e.g., from strains 129/SV, C57/BL6, DBA-1, 129/SVJ), K562, Jurkat cells, BW5147 and any other commercially available human cell lines. Other useful mammalian cell lines are well known and readily available from the American Type Culture Collection (ATCC) (Manassas, Va., USA) and the National Institute of General Medical Sciences (NIGMS) Human Genetic Cell Repository at the Coriell Cell Repositories (Camden, N.J., USA). Exemplary prokaryotic host cells include, for example, E. coli.

In a further aspect, certain embodiments provide pharmaceutical compositions comprising SP-CHAP or any of the SP-CHAP peptide fragments and variants described herein, e.g., for use in any of the therapeutic methods used for treatment of pneumococcal disease. In one embodiment, a pharmaceutical composition comprising a SP-CHAP and a pharmaceutically acceptable carrier is provided herein. Expression vectors expressing SP-CHAP may also be used for pneumococcal disease. Such vectors may be combined with a pharmaceutically acceptable carrier. In another embodiment, a pharmaceutical composition comprises the SP-CHAP provided herein and at least one additional therapeutic agent, typically used for treatment of pneumococcal disease.

The present disclosure further provides nanoparticles comprising SP-CHAP reagents as disclosed herein. Such nanoparticles can be natural or synthetic and may be incorporated into an SP-CHAP composition. They can be created from biological molecules or from non-biological molecules. In some cases, the SP-CHAP reagents are crosslinked to a polymer or lipid on the nanoparticle surface. In embodiments, the SP-CHAP reagents are adsorbed onto the nanoparticle surface. In some embodiments, the SP-CHAP reagents are adsorbed onto the nanoparticle surface and then crosslinked to the nanoparticle surface. In some embodiments, the SP-CHAP reagents are encapsulated into the nanoparticle.

In particular embodiments, the nanoparticle is formed from a biocompatible polymer. Examples of biocompatible polymers include polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, or combinations thereof. In some cases, the nanoparticle is formed from a polyethylene glycol (PEG), poly(lactide-co-glycolide) (PLGA), polyglycolic acid, poly-beta-hydroxybutyrate, polyacrylic acid ester, or a combination thereof.

In a specific embodiment the nanoparticle is a nanoliposome. Such nanoliposomes may be composed of phospholipids such as 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1,2-distearoyl-sn-glycero-3-phospho-(1′-rac-glycerol) (DSPG), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1,2-dioleoyl-sn-glycero-3-phospho-(1′-rac-glycerol) (DOPG), dipalmitoyl phosphatidylserine (DPPS), distearoyl phosphatidylserine (DSPS), dipalmitoyl phosphatidylinositol (DPPI), distearoyl phos phatidylinositol (DSPI), dipalmitoyl phosphatidic acid (DPPA), distearoyl phosphatidic acid (OSPA), 1,2-diacyl-3-trimethylammonium-propanes, (including but not limited to, dioleoyl (DOTAP), 1,2-dipalmitoyl-sn-glycero-3-phosphoethanolamine-N [methoxy (polyethylene glycol)-(DPPE-PEG2000], 1,2-distearoyl-sn-glycero-3-phosphoethanolamine-N-[methoxy (polyethylene glycol)-1000] (DSPE-PEG2000), and cholesterol.

In some embodiments, the SP-CHAP reagents are coated on the nanoparticle using a crosslinking agent. In some embodiments, the SP-CHAP reagents are adsorbed onto the nanoparticle surface. In some embodiments, the SP-CHAP reagents are adsorbed onto the nanoparticle surface followed by covalent crosslinking of the SP-CHAP reagents to the nanoparticle surface using a crosslinking agent. Crosslinking agents suitable for crosslinking are known in the art.

In some embodiments, the SP-CHAP reagents are encapsulated into the nanoparticle. Useful delivery vectors for inclusion of the SP-CHAP compositions include biodegradable microcapsules, immuno-stimulating complexes (ISCOMs) or liposomes. Liposome vectors may also be used for delivery of nucleic acids or proteins. Such liposome vectors may be unilamellar or multilamellar vesicles, having a membrane portion formed of lipophilic material and an interior aqueous portion. The aqueous portion is used to contain the polynucleotide material to be delivered to the target cell. In general, the liposome forming materials have a cationic group, such as a quaternary ammonium group, and one or more lipophilic groups, such as saturated or unsaturated alkyl groups having about 6 to about 30 carbon atoms. One group of suitable materials is described in European Patent Publication No. 0187702, and further discussed in U.S. Pat. No. 6,228,844 to Wolff et al., the pertinent disclosures of which are incorporated by reference. Many other suitable liposome-forming cationic lipid compounds are described in the literature. See, e.g., L. Stamatatos, et al., Biochemistry 27:3917 3925 (1988); and H. Eibl, et al., Biophysical Chemistry 10:261 271 (1979). Alternatively, a microsphere such as a polylactide-co-glycolide biodegradable microsphere can be utilized. A SP-CHAP encoding nucleic acid is encapsulated or otherwise complexed with the liposome or microsphere for delivery of the nucleic acid to a tissue, as is known in the art.

Further provided is a method of producing a SP-CHAP of certain embodiments in a form suitable for administration in vivo, the method comprising (a) obtaining SP-CHAP according to any of the various embodiments disclosed above for SP-CHAP production, and (b) formulating the SP-CHAP with at least one pharmaceutically acceptable carrier, whereby a preparation of the SP-CHAP is formulated for administration in vivo.

Further provided is a method of producing a SP-CHAP encoding nucleic acid of certain embodiments in a form suitable for administration in vivo, the method comprising (a) obtaining a SP-CHAP encoding nucleic acid according to various embodiments, and (b) formulating the SP-CHAP encoding nucleic acid with at least one pharmaceutically acceptable carrier, whereby a preparation of the SP-CHAP encoding nucleic acid is formulated for administration in vivo.

In a specific embodiment, a mRNA-based approach may be utilized to deliver SP-CHAP to the site of infection. Such administration of SP-CHAP encoding nucleic acids (“mRNA delivery system”) enables endogenous synthesis and production of the endolysin within target organs or tissues. Contact of SP-CHAP RNA to cells, such as human cells, would result in the expression and production of SP-CHAP in the cytosol of the contacted cell.

As an example, the provided mRNA delivery system can be administered directly to the tissue where bacterial colonization or infection is prevalent. In one embodiment, an aerosolized delivery of mRNA could be used to transfect lung epithelial cells, which, in turn, would secrete the manufactured SP-CHAP resulting in localized anti-bacterial activity. In some instances, the SP-CHAP mRNA can be administered directly to surgical wounds or prosthetic joints to lessen the chance of post-surgical infection.

In some embodiments, the mRNA expressing constructs are optimized for cellular expression. For example, a signal peptide (SP) sequence may be attached to the N-terminus of the SP-CHAP encoding sequence so as to enable the secretion of the endolysin form the cell. Additionally, mutations may be engineered into the SP-CHAP encoding sequence to prevent modification of the protein, e.g., glycosylation. In one embodiment, the 5′ and 3′ untranslated regions (UTRs) of the RNA can be stabilized with human alpha-globin UTRs, a 5′ cap-1 structure can be included and/or a poly(A) tail of at least 120 base pairs can be added to the 3′end of the RNA.

In some embodiments, the disclosed SP-CHAP and/or compositions including the SP-CHAP as disclosed herein may be coupled to the surface of a substrate. For example, in some implementations, a medical device (e.g., a grasper, a clamp, a retractor, a dilator, a suction, a sealing device, a scope, a probe, etc.) includes an outer surface coupled to or coated with the SP-CHAP or composition comprising the SP-CHAP. In some implementations, the medical device coupled to or coated with the SP-CHAP or compositions comprising the SP-CHAP is an implantable medical device (e.g., a drainage tube, a feeding tube, a shunt, a prosthesis, a guidance tube, a catheter, a valve, a pacemaker, a graft, a tissue scaffold, a stent, etc.).

The SP-CHAP disclosed herein, and compositions comprising such polypeptides, are also suitable for use as a sanitizing agent or disinfectant of a target surface or area. Thus, methods and compositions are provided for treating or preventing bacterial contamination of dental and medical devices, surfaces in hospitals and dental and medical facilities, food processing equipment, surfaces in food processing facilities, equipment and surfaces in schools, and other equipment or surfaces on which sanitization is desired.

In addition, the SP-CHAP compositions may be used in combination with other disinfecting ingredients, cleaners, and agents (e.g., such as detergents, solvents, antibiotics, antimicrobials, etc.). In some implementations, SP-CHAP compositions are applied to target surfaces or areas as a liquid or spray formulation (e.g., aerosolized or mist formulation). Disclosed compositions may be applied, e.g., with a dry mist fogger or other such application, for disinfecting surfaces within a target area or volume (e.g., a milking parlor, school gymnasium or auditorium, surgical suite, medical equipment, etc.).

Pharmaceutical compositions of embodiments comprise a therapeutically effective amount of SP-CHAP and/or SP-CHAP nucleic acids (“SP-CHAP reagents”) dissolved or dispersed in a pharmaceutically acceptable carrier. The preparation of a pharmaceutical composition that contains SP-CHAP reagents and optionally an additional active ingredient will be known to those of skill in the art in light of the present disclosure, as exemplified by Remington's Pharmaceutical Sciences, 18th Ed. Mack Printing Company, 1990. For human administration, it will be understood that preparations should meet sterility, pyrogenicity, general safety and purity standards as required by FDA Office of Biological Standards or corresponding authorities in other countries. Preferred compositions are lyophilized formulations or aqueous solutions.

As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it needs to be sterile for such routes of administration as injection. SP-CHAP of certain embodiments (and any additional therapeutic agent) can be administered by any method or any combination of methods as would be known to one of ordinary skill in the art (see, for example, Remington's Pharmaceutical Sciences, 18th Ed. Mack Printing Company, 1990, incorporated herein by reference). Parenteral administration may be used for administering protein or polypeptide molecule such as the SP-CHAP of certain embodiments. Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Parenteral compositions include those designed for administration by injection, e.g. subcutaneous, intradermal, intra-lesional, intravenous, intra-arterial, intramuscular, intrathecal or intraperitoneal injection. For injection, the SP-CHAP may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the SP-CHAP may be in powder form for constitution with a suitable vehicle, e.g., sterile pyrogen-free water, before use. Sterile injectable solutions are prepared by incorporating the SP-CHAP in the required amount in the appropriate solvent with various other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi.

Pharmaceutical compositions comprising SP-CHAP reagents may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

SP-CHAP may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The pharmaceutical preparation of certain embodiments is a liquid composition, e.g. an aqueous solution. For injection purposes, the use of pure water as solvent is preferred. Other solvents which are suitable and conventional for pharmaceutical preparations can, however, also be employed. In a preferred embodiment, the pharmaceutical compositions are isotonic solutions. Further, there is no need for reconstitution at any stage of the preparation of the liquid solution formulation of these embodiments. The solution is a ready-to-use formulation.

The delivery of a therapeutic SP-CHAP to appropriate cells can occur via gene therapy ex vivo, in situ, or in vivo by use of any suitable approach known in the art. For example, for in vivo therapy, a nucleic acid encoding the desired SP-CHAP, either alone or in conjunction with a vector, liposome, or precipitate may be injected directly into the subject, and in some embodiments, may be injected at the site where the expression of the SP-CHAP is desired, e.g., the infection site. In another embodiment, the SP-CHAP is delivered intranasally. For ex vivo treatment, the subject's cells are removed, the nucleic acid is introduced into these cells, and the modified cells are returned to the subject either directly or, for example, encapsulated within porous membranes which are implanted into the patient. See, e.g. U.S. Pat. Nos. 4,892,538 and 5,283,187.

A variety of techniques are available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, chemical treatments, DEAE-dextran, and calcium phosphate precipitation. Other in vivo nucleic acid transfer techniques include transfection with viral vectors (such as adenovirus, Herpes simplex I virus, adeno-associated virus, lentivirus or retrovirus) and lipid-based systems. The nucleic acid and transfection agent are optionally associated with a microparticle. Exemplary transfection agents include calcium phosphate or calcium chloride co-precipitation, DEAE-dextran-mediated transfection, quaternary ammonium amphiphile DOTMA ((dioleoyloxypropyl)trimethylammonium bromide, commercialized as Lipofectin by GIBCO-BRL)) (Felgner et al, (1987) Proc. Natl. Acad. Sci. USA 84, 7413-7417; Malone et al. (1989) Proc. Natl Acad. Sci. USA 86 6077-6081); lipophilic glutamate diesters with pendent trimethylammonium heads (Ito et al. (1990) Biochem. Biophys. Acta 1023, 124-132); the metabolizable parent lipids such as the cationic lipid dioctadecylamido glycylspermine (DOGS, Transfectam, Promega) and dipalmitoylphosphatidyl ethanolamylspermine (DPPES) (J. P. Behr (1986) Tetrahedron Lett. 27, 5861-5864; J. P. Behr et al. (1989) Proc. Natl. Acad. Sci. USA 86, 6982-6986); metabolizable quaternary ammonium salts (DOTB, N-(1-[2,3-dioleoyloxy]propyl)-N,N,N-trimethylammonium methylsulfate (DOTAP) (Boehringer Mannheim), polyethyleneimine (PEI), dioleoyl esters, ChoTB, ChoSC, DOSC) (Leventis et al. (1990) Biochim. Inter. 22, 235-241); 3 beta [N—(N′,N′-dimethylaminoethane)-carbamoyl]cholesterol (DC-Chol), dioleoylphosphatidyl ethanolamine (DOPE)/3beta [N—(N′,N′-dimethylaminoethane)-carbamoyl]cholesterolDC-Chol in one-to-one mixtures (Gao et al., (1991) Biochim. Biophys. Acta 1065, 8-14), spermine, spermidine, lipopolyamines (Behr et al., Bioconjugate Chem, 1994, 5:382-389), lipophilic polylysines (LPLL) (Zhou et al., (1991) Biochim. Biophys. Acta 939, 8-18), [[(1, 1, 3, 3-tetramethylbutyl) cre-soxy]ethoxy]ethyl]dimethylbenzylammonium hydroxide (DEBDA hydroxide) with excess phosphatidylcholine/cholesterol (Ballas et al., (1988) Biochim. Biophys. Acta 939, 8-18), cetyltrimethylammonium bromide (CTAB)/DOPE mixtures (Pinnaduwage et al, (1989) Biochim. Biophys. Acta 985, 33-37), lipophilic diester of glutamic acid (TMAG) with DOPE, CTAB, DEBDA, didodecylammonium bromide (DDAB), and stearylamine in admixture with phosphatidylethanolamine (Rose et al., (1991) Biotechnique 10, 520-525), DDAB/DOPE (TransfectACE, GIBCO BRL), and oligogalactose bearing lipids. Exemplary transfection enhancer agents that increase the efficiency of transfer include, for example, DEAE-dextran, polybrene, lysosome-disruptive peptide (Ohmori N I et al, Biochem Biophys Res Commun Jun. 27, 1997; 235 (3): 726-9), chondroitan-based proteoglycans, sulfated proteoglycans, polyethylenimine, polylysine (Pollard H et al. J Biol Chem, 1998 273 (13): 7507-11), integrin-binding peptide CYGGRGDTP (SEQ ID NO: 10), linear dextran nonasaccharide, glycerol, cholesteryl groups tethered at the 3′-terminal internucleoside link of an oligonucleotide (Letsinger, R. L. 1989 Proc Natl Acad Sci USA 86: (17): 6553-6), lysophosphatide, lysophosphatidylcholine, lysophosphatidylethanolamine, and 1-oleo yl lysophosphatidylcholine.

In some situations, it may be desirable to deliver the nucleic acid with an agent that directs the nucleic acid-containing vector to target cells. Such “targeting” molecules include antigen binding proteins specific to a cell-surface membrane protein on the target cell, or a ligand for a receptor on the target cell. In a specific embodiment, the targeted cells may be lung cells. Where liposomes are employed, proteins which bind to a cell-surface membrane protein associated with lung cells, for example, may be used for targeting and/or to facilitate uptake. For review of the currently known gene marking and gene therapy protocols, see Anderson 1992. See also WO 93/25673 and the references cited therein. For additional reviews of gene therapy technology, see Friedmann, Science, 244:1275-1281 (1989); Anderson, Nature, supplement to vol. 392, no 6679, pp. 25-30 (1998); Verma, Scientific American: 68-84 (1990); and Miller, Nature, 357:455460 (1992).

Any of the SP-CHAP reagents provided herein may be used in therapeutic methods described herein. For use in the therapeutic methods described herein, SP-CHAP encoding nucleic acids, of certain embodiments would be formulated, dosed, and administered in a fashion consistent with good medical practice. Factors for consideration in this context include the particular disorder being treated, the particular subject being treated, the clinical condition of the subject, the cause of the disease or condition, the site of delivery of the agent, the method of administration, the scheduling of administration, and other factors known to medical practitioners or those of skill in the art. In certain embodiments the method further comprises administering to the individual a therapeutically effective amount of at least one additional therapeutic agent, e.g. an agent that is typically used to treat the pneumococcal disease to be treated.

For the treatment of pneumococcal disease, the appropriate dosage of SP-CHAP (when used alone or in combination with one or more other additional therapeutic agents) will depend on the type of disease to be treated, the route of administration, the body weight of the patient, the severity and course of the disease, whether the SP-CHAP is administered for preventive or therapeutic purposes, previous or concurrent therapeutic interventions, the patient's clinical history and response to the SP-CHAP, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

SP-CHAPs are suitably administered to the patient at one time or over a series of treatments subcutaneously, intravenously, intramuscularly, in the ear, locally or via airway or under tongue. In one aspect, SP-CHAP is administered directly to surgical wounds or prosthetic joints to lessen the chance of post-surgical infection. For repeated administration over several days or longer, depending on the condition, the treatment would generally be sustained until a desired suppression of disease symptoms occurs. In some instances, the SP-CHAP composition may be freeze dried or lyophilized such that it can be administered through a nebulizer or inhaler for delivery to the lungs.

Pneumococcal bacterium can form biofilms to promote colonization of the nasopharynx and to improve antibiotic resistance. As disclosed herein, SP-CHAP is highly efficient at eradicating said biofilms. Accordingly, in a specific embodiment, the compositions disclosed herein are formulated for intranasal administration. Intranasal administration of the compositions, if used, is generally characterized by inhalation. Compositions for nasal administration can be prepared so that, for example, the SP-CHAP can be administered directly to the mucosa (e.g., nasal and/or pulmonary mucosa).

Optionally, such intranasal compositions may further advantageously comprise a mucoadhesive, such as cellulose derivatives, polyacrylates, starch, chitosan, glycosaminoglycans, hyaluronic acid, and any combination thereof. The mucoadhesive may be present in the composition at about 0.1% to about 10% by weight. For example, the composition can be formulated for intranasal delivery as a dry powder, as an aqueous solution, an aqueous suspension, a colloidal suspension, a water-in-oil emulsion, a micellar formulation, or as a liposomal formulation.

Methods for mucosal delivery include those methods known in the art that provide delivery of the composition to mucous membranes. Mucosal delivery methods include intranasal, intrabuccal, and oral. In some embodiments, the administration is intranasal. In these embodiments, the anti-bacterial composition may be formulated to be delivered to the nasal passages or nasal vestibule of the subject as droplets, an aerosol, micelles, lipid or liquid nanospheres, liposomes, lipid or liquid microspheres, a solution spray, or a powder. The composition can be administered by direct application to the nasal passages or may be atomized or nebulized for inhalation through the nose or mouth.

In some embodiments, the method comprises administering a nasal spray, medicated nasal swab, medicated wipe, nasal drops, or aerosol to the subject's nasal passages or nasal vestibule. In some embodiments, the compositions of present invention can be delivered using a nasal spray device, which can allow (self) administration with little or no prior training to deliver a desired dose. The apparatus can comprise a reservoir containing a quantity of the composition. The apparatus may comprise a pump spray for delivering one or more metered doses to the nasal cavity of a subject. The device may advantageously be single dose use or multi-dose use. It further may be designed to administer the intended dose with multiple sprays, e.g., two sprays, e.g., one in each nostril, or as a single spray, e.g., in one nostril, or to vary the dose in accordance with the body weight or maturity of the patient. In some embodiments, nasal drops may be prepacked in pouches or ampoules that may be opened immediately prior to use and squeezed or squirted into the nasal passages. In some embodiments, where the bacterial infection has spread to the ear, the SP-CHAP may be included in compositions designed for delivery to the ear. Such compositions include, for example, ear drops.

Non-limiting typical dosages may be in the range from about 1 μg/kg body weight to 1000 mg/kg body weight. In other non-limiting examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein.

Such doses may be administered intermittently, e.g. every week or every three weeks (e.g. such that the patient receives from about two to about twenty, or e.g. about six doses of the SP-CHAP). An initial higher loading dose, followed by one or more lower doses, may be administered. However, other dosage regimens may be useful. The progress of this therapy is easily monitored by conventional techniques and assays. The SP-CHAPs of certain embodiments will generally be used in an amount effective to achieve the intended purpose. For use to treat or prevent a disease condition, the SP-CHAP of these embodiments, or pharmaceutical compositions thereof, are administered or applied in a therapeutically effective amount. Determination of a therapeutically effective amount is well within the capabilities of those skilled in the art, especially in light of the detailed disclosure provided herein.

For the administration of SP-CHAP, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

The SP-CHAP containing compositions may be administered by an initial bolus followed by a continuous infusion to maintain therapeutic circulating levels of drug product. As another example, the inventive compound may be administered as a one-time dose. Those of ordinary skill in the art will readily optimize effective dosages and administration regimens as determined by good medical practice and the clinical condition of the individual patient.

Sustained-release preparations may be prepared. Suitable examples of sustained-release preparations include semi-permeable matrices of solid hydrophobic polymers containing the immunoglobulin, which matrices are in the form of shaped articles, e.g., films, or microcapsule. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable the release of molecules for over 100 days, certain hydrogels release proteins for shorter time periods. When encapsulated polypeptides remain in the body for a long time, they may denature or aggregate because of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The formulations of other embodiments may be designed to be short-acting, fast-releasing, long-acting, or sustained-releasing as described herein. Thus, the pharmaceutical formulations may also be formulated for controlled release or for slow release.

The attending physician for patients treated with SP-CHAP of certain embodiments would know how and when to terminate, interrupt, or adjust administration due to toxicity, organ dysfunction, and the like. Conversely, the attending physician would also know how to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient. The SP-CHAP described herein may be administered in combination with one or more other agents or “therapeutic agents” for use in the treatment of pneumococcal disease. A SP-CHAP may be co-administered with at least one additional therapeutic agent. The term “therapeutic agent” encompasses any agent administered to treat a symptom or disease in an individual in need of such treatment. Such an additional therapeutic agent may comprise any active ingredients suitable for the particular indication being treated, preferably those with complementary activities that do not adversely affect each other. Such therapeutic agents include for example, bacterial antibiotics such as amoxicillin, clarithromycin, and levofloxacin, pain relievers and cough suppressants to name a few.

During Streptococcus pneumoniae nasopharyngeal colonization, the activation of programmed necrosis, specifically necroptosis, plays a critical role in the development of antigen-specific antibodies against the pathogen. The treatment of colonized mice with endolysins, such as SP-CHAP, not only effectively eradicates colonizing bacteria but also significantly enhances necroptosis activation, thus indicating that endolysin treatment can act as an immunological catalyst, amplifying the development of protective immunity against S. pneumoniae. By using the lytic activity of endolysins, bacterial antigens are released from the colonizing pathogens and made available for presentation to the host immune system.

Accordingly, the present disclosure provides SP-CHAP containing compositions that function as novel vaccine adjuvants or potentiator, (“SP-CHAP vaccine”), transforming the act of bacterial clearance into a mechanism for driving a robust immune response against bacterial components. In an embodiment, the immune response is a serotype-independent immune response. In another embodiment, the SP-CHAP vaccine compositions for use in stimulation of an anti-bacterial reaction may include adjuvants know in the art to stimulate an immune response. Suitable adjuvants include Freund's complete and incomplete adjuvant, Titermax, oil in water adjuvants, as well as aluminum compounds. In general, the SP-CHAP vaccines will comprise a sufficient amount of SP-CHAP to induce an immune response against the bacteria.

In one aspect, the present disclosure is directed to methods of generating an immune response in a subject to a SP-CHAP vaccine formulation as disclosed herein. In one embodiment, the present disclosure is directed to methods of generating an immune response to Streptococcus pneumoniae in a subject, comprising administering an immunologically effective amount of a vaccine formulation of the present disclosure to a subject, thereby generating an immune response against said bacteria in a subject. In each of the methods of generating an immune response of the present disclosure, the immune response is preferably a protective immune response.

An “immunologically effective amount” of a vaccine formulation is one that is sufficient to induce an immune response to vaccine components in the subject to which the vaccine formulation is administered. A “protective immune response” is one that confers on the subject to which the vaccine formulation is administered protective immunity against the pneumococcal bacteria. The protective immunity may be partial or complete immunity.

The vaccine formulations of the present disclosure may also be used in methods of inhibiting a pneumococcal bacteria infection in a subject. Such methods comprise administering a therapeutically effective amount of a vaccine formulation of the present disclosure to a subject at risk of developing a pneumococcal bacterial infection, thereby inhibiting a pneumococcal bacterial infection in a subject. In a preferred embodiment, the method further comprises administering an anti-bacterial agent to the subject at risk of developing a pneumococcal bacterial infection in conjunction with the administration of the vaccine formulation.

Administration of the vaccine formulations may be via any of the means commonly known in the art of vaccine delivery. Such routes include intravenous, intraperitoneal, intramuscular, subcutaneous and intradermal routes of administration, as well as nasal application, by inhalation, to the ear, ophthalmically, orally, rectally, vaginally, or by any other mode that results in the vaccine formulation contacting mucosal tissues.

As a specific example, the SP-CHAP vaccine formulations exist as atomized dispersions for delivery by inhalation. The atomized dispersion of the SP-CHAP vaccine formulation typically contains carriers common for atomized or aerosolized dispersions, such as buffered saline and/or other compounds well known to those of skill in the art. The delivery of the SP-CHAP vaccine formulations via inhalation has the effect of rapidly dispersing the vaccine formulation to a large area of mucosal tissues. One example of a method of preparing an atomized dispersion is described in U.S. Pat. No. 6,187,344, entitled, “Powdered Pharmaceutical Formulations Having Improved Dispersibility,” which is hereby incorporated by reference in its entirety.

In another aspect of the embodiment, an article of manufacture (e.g., a kit) containing materials useful for the treatment of pneumococcal disease as described above is provided. The article of manufacturing comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle).

The label or package insert indicates that the composition is used for treating the condition of choice. The article of manufacture may comprise (a) a first container with a composition contained therein, wherein the composition comprises SP-CHAP; and (b) a second container with a composition contained therein, wherein the composition comprises a further therapeutic agent.

Kits in certain embodiments may further comprise a package insert indicating that the compositions can be used to treat a particular condition. Alternatively, or additionally, the kit may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that the examples and embodiments described herein are for illustrative purposes only and that various modifications or changes in light thereof will be suggested to persons skilled in the art and are to be included within the spirit and purview of this application and scope of the appended claims.

All documents, papers and published materials referenced herein, including books, journal articles, manuals, patent applications, published patent applications and patents, are expressly incorporated herein by reference in their entireties.

### EXAMPLES

### Material and Methods

### Bacterial Strains and Growth Conditions

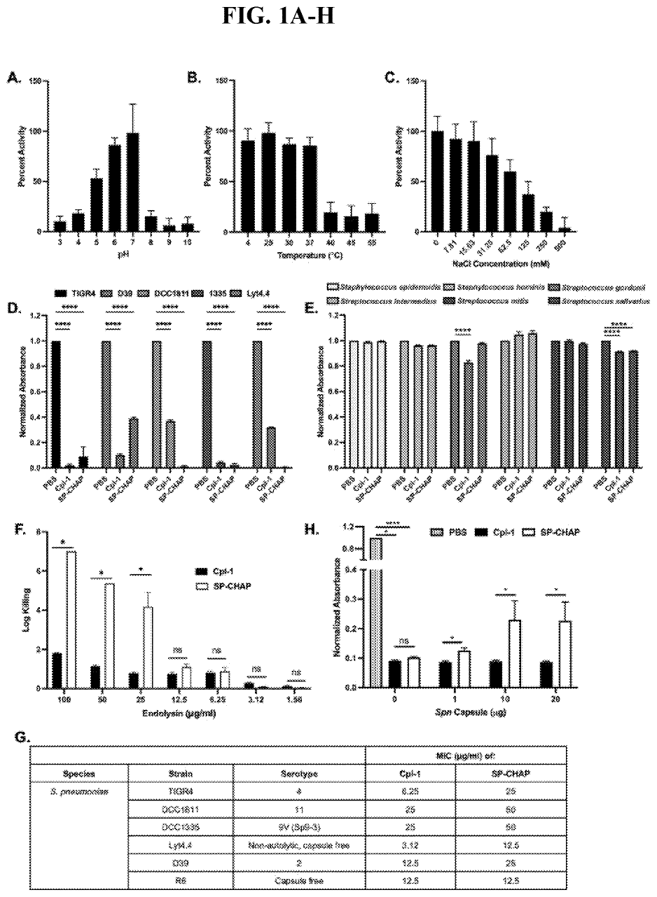

Streptococcus pneumoniae was grown in static culture at 37° C. with 5% CO₂ in Todd Hewitt broth supplemented with 1% yeast extract (THY) or on THY agar plates. The Spn strain R6 is a capsule-free variant derived from strain D39, a serotype 2 strain (FIG. 1G). Strain Lyt4.4 represents a transformed version of the R6 strain where the major autolysin, LytA, has been inactivated, as previously described (22). Thus, Lyt4.4 is both capsule-free and LytA deficient. Due to the autolytic activity that Spn normally display in overnight cultures, in biofilms, and when exposed to capsule shedding, Spn Lyt4.4 was used for these experiments. Commensal organisms, Staphylococcus hominis SK119, Staphylococcus epidermidis SK135, Streptococcus mitis F0392, Streptococcus gordonii PK2565, Streptococcus intermedius PK2821, and Streptococcus salivarius ATCC 27945 were grown in Brain Heart Infusion media (Difco) at 37° C. Escherichia coli DH5a and BL21 (DE3) strains were grown in Luria-Bertani (LB) broth or agar. All strains were stored at −80° C.

### Cloning

Cpl-1 (GenBank: NP_044837.1) and SP-CHAP (OR644363) used in this study were codon-optimized for E. coli expression and synthesized (GeneArt). A 6×-His tag was added to the C-terminal end. The constructs were sub-cloned into the pBAD24 expression vector, and the sequence was confirmed (Psomagen, Rockville, MD, USA) before being transformed into E. coli BL21 (DE3) for protein expression. The ApE program (University of Utah) was used for DNA sequence analysis and manipulations.

### Protein Expression and Purification

Overnight cultures of E. coli BL21 (DE3) containing the plasmid of interest were sub-cultured 1:100 into 1.5 L baffled flasks containing LB supplemented with 100 μg/mL antibiotic and grown at 37° C. Expression was induced while cells were in the mid-log phase with 0.25% arabinose and incubated overnight at 18° C. Cells were harvested (5,000×g, 15 min, 4° C.), resuspended in 20 mL of lysis buffer (PBS and 10 mM imidazole, pH 7.4), and disrupted by freeze-thawing (−80° C.-4° C.). This was followed by sonication on ice (power level 6 and duty cycle 30%). Insoluble cell debris was removed by centrifugation (12,000× g, 1 h, 4° C.), and the cell lysate containing the 6×-His tagged protein was loaded onto Ni-NTA resin (HisPur Ni-NTA Resin, Thermo Scientific) in a gravity flow column and eluted in 4 mL fractions using an imidazole step gradient in PBS buffer (20, 50, 100, 250, and 500 mM imidazole). Protein purity was analyzed on an SDS-PAGE gel. Samples were additionally purified using a Sephacryl S-200 column and an ÄKTA Pure 25 L system (both from Cytiva). Samples were pooled and concentrated using a protein concentrator with a 10 kDa molecular weight cutoff (Amicon Ultra 15 mL centrifugal filters, Millipore Sigma).

### Turbidity Reduction Assay

Bacteriolytic activity was measured by means of a turbidity reduction assay as previously described (23). Overnight cultures of Spn were centrifuged, and the pellets were washed twice and resuspended in PBS to a final optical density at 600 nm (OD600) of 0.9-1.2. Cells were mixed with SP-CHAP or Cpl-1 with a final protein concentration of 100 μg/mL. The OD600 was monitored by the use of a microplate spectrophotometer (SpectraMax ABS; Molecular Devices, USA) every 15 s for 30 min at 37° C. Lytic activity was quantified as the reduction in turbidity and measured as the difference in OD600 between PBS control-treated cells and cells that were treated with SP-CHAP or Cpl-1 over 30 min.

### Colony-Forming Unit Assay

CFU counting was also conducted to quantify antimicrobial activity. Sterile protein at a final concentration of 100 μg/mL was used for this assay. Briefly, 100 μL of protein was mixed with 100 μL of Spn Lyt4.4 in PBS in a 96-well plate and incubated at 37° C. for an hour. Then, five 10-fold dilutions in PBS were made, and 10 μL from each dilution was plated on THY agar plates, air-dried, and placed in a 37° C. incubator overnight. The CFUs were counted, and the data were reported as the log-fold killing compared to the untreated control. Each turbidity reduction assay and CFU counting assay were performed in triplicate on three separate days.

### Minimum Inhibitory Concentration Assay

One hundred microliters of an overnight Spn culture was diluted in 10 mL of THY media (1:100) with 50 μM catalase. Ten twofold dilutions of endolysins were prepared, starting at 100 μg/mL. Following this, 100 μL of the diluted culture was mixed with 100 μL of each endolysin treatment at every concentration in triplicate, using a 96-well plate. The plate was then incubated overnight in a 37° C. non-shaking incubator and observed the following day to identify the lowest concentration at which there was a clear well with no bacterial growth. The OD600 was measured for accuracy using a SpectraMax ABS plate reader.

### Endolysin Inhibition Assay

Overnight cultures of Spn Lyt4.4 were centrifuged, and the pellets were washed twice and resuspended in PBS. SP-CHAP or Cpl-1 with a final protein concentration of 50 μg/mL were mixed with 1, 10, or 20 μg of purified Spn serotype 4 capsular polysaccharide (76855, SSI Diagnostica) and incubated at room temperature for 15 min. Spn cultures were then challenged with the endolysin-capsule mixtures, and after a 30-min incubation time, lytic activity was quantified via the turbidity reduction assay described above.

### Biofilm Assay

Spn Lyt4.4 were grown in THY media to an OD600 of 0.5-0.6, sedimented by centrifugation, and resuspended in an equal volume of fresh THY media. The cells were then diluted 1:2 into a fresh 15 mL tube, and 200 μL of this was dispensed into each well of a Costar 96-well flat-bottom polystyrene plate (Corning). The plate was then incubated at 37° C. overnight. The next day, the media were carefully pipetted out from each well, and the biofilm formed was gently washed twice with PBS to remove unattached cells. Twofold serial dilutions of SP-CHAP or Cpl-1 were added to treatment wells, and the plate was incubated at 37° C. for 1 h. The liquid was then pipetted out, and the wells were washed with distilled water and left to air dry. Biofilms were stained with 50 L of a 0.2% crystal violet solution for 15 min and subsequently washed three times with distilled water and air-dried. The biofilm was solubilized in 10% SDS to extract crystal violet from the biomass, transferred to a fresh 96-well plate, and absorbance quantified at OD595 using a SpectraMax M5 plate reader. Alternately, identically treated biofilms with 20 μg/mL Cpl-1 or SP-CHAP were scrapped from wells, pipetted to break up the biofilms, serially diluted, and incubated overnight as described above in the colony-forming unit assay to enumerate Spn viability.

### Confocal Microscopy

Spn biofilms were grown in chamber slides (Nunc Lab-Tek chamber slides, Thermo Fisher Scientific) following previously established procedures (24). One hundred microliters of a 100 μg/mL solution of each endolysin was added and allowed to incubate at room temperature for 1 h. After washing with PBS, the biofilms were stained using the Live/Dead BacLight stain (Thermo Fisher Scientific) before being examined by confocal microscopy. Mounted samples were visualized using a Zeiss LSM800 confocal microscope at 63× with 1× digital zoom. Z-stacks of each biofilm sample were acquired using the 488 nm laser and 500-619 nm detection filter with a constant 0.5 μm Z-axis interval for 19 slices. 3D images of the biofilms were rendered using the normal shading mode of Imaris 9.0.1 software (Oxford Instruments). All acquisition and image settings were set the same across all samples.

### Acute Systemic Toxicity Testing

Acute systemic toxicity testing was done by Pacific BioLabs. Briefly, SP-CHAP was prepared according to ISO 10993-12 and Pacific BioLabs internal SOPs (Pacific BioLabs, Hercules, CA, USA). Saline without SP-CHAP was used as the vehicle (negative) control. Ten Swiss-Webster albino mice were randomly assigned to SP-CHAP or vehicle groups. Each animal was injected intraperitoneally with 200 μL of saline or SP-CHAP at a concentration of 3 mg/mL. The animals were observed for biological reactivity immediately after and at 4 h±15 min, 24±2 h, 48±2 h, and 72±2 h following injection. Animals were weighed at 24±2 h, 48±2 h, and 72±2 h following injection. At the end of the study, animals were euthanized, and gross necropsy was performed.

### Mouse Colonization Studies

The mouse colonization experiments were similar to those previously described (20, 25). Briefly, mice were infected intranasally with 1×10⁵ CFU Spn TIGR4. After 48 h, 25 μL of endotoxin-free, filter-sterilized PBS (vehicle), 1.2 mg/mL Cpl-1, or 1.2 mg/mL SP-CHAP were delivered to each nostril. After an additional 4 h, mice were euthanized and nasopharyngeal tissue was excised, weighed, and homogenized. Homogenates were serially diluted and plated on blood agar plates to determine CFU per gram of tissue.

### Statistical Analysis

Unless otherwise noted, all in vitro experiments had a minimum of three biological replicates, with ≥3 technical replicates. GraphPad Prism 8 (La Jolla, CA, USA) was used for statistical analyses. Comparisons between two cohorts at a single time point were calculated by the Mann-Whitney U test. Comparisons between groups of >2 cohorts or groups given multiple treatments were calculated by ANOVA with Tukey's (one-way) or Dunnett's (two-way) post-test or by Kruskal-Wallis H test with Dunn's multiple comparison post-test, as determined by the normality of data groups. Repeated measures are accounted for whenever applicable.

### Results

Endolysins offer an alternative to conventional antibiotics in addressing the emergence of multi-drug-resistant bacteria. These enzymes, encoded by bacteriophages, induce cell lysis upon synthesis at the end of the replication cycle and are not susceptible to efflux pumps, penicillin-binding proteins, or other common mechanisms of antimicrobial resistance (1, 2). Moreover, the development of any resistance to endolysins has not been reported (3). Streptococcus pneumoniae (Spn), a Gram-positive nasopharyngeal pathobiont, can cause infections, including pneumonia, invasive pneumococcal disease, meningitis, and sepsis (4, 5). As novel antibiotic approval rates have declined and the frequency of antibiotic-resistant strains of Spn has surged, addressing this pathogen is imperative (6). Several endolysins that target Spn, such as Cpl-1 (7) and Pal (8), have shown efficacy both in vitro and in vivo. Many endolysins share a common cysteine, histidine-dependent amidohydrolase/peptidase (CHAP) domain (9). Several CHAP-containing endolysins with antimicrobial activity against Staphylococcus aureus, including N-Rephasin (SAL200) and Exebacase (CF-301), have been investigated in human clinical trials (3). However, no naturally occurring pneumococcal endolysin with a CHAP domain has been described. 76 putative pneumococcal endolysins have recently been identified from uncultured bacteriophage genomes by searching for a consensus sequence found in the cell-binding domain region of Cpl-1, Pal, and LytA (a Spn autolysin). One candidate endolysin, SP-CHAP, contains a CHAP domain and forms a dimer in the presence of choline, suggesting increased binding to the pneumococcal cell wall (10). Set forth below is a characterization of the biochemical and antimicrobial properties of SP-CHAP and a benchmark against Cpl-1.

### Biochemical Characterization of SP-CHAP

To determine the optimum pH for SP-CHAP enzymatic activity, lytic activity over a pH range of 3.0-10.0 in a turbidity reduction assay was measured (FIG. 1A). The optimum SP-CHAP pH of 6.0-7.0 is similar to that seen for Cpl-1, Pal, and LytA (11). Next, the thermodynamic stability of SP-CHAP was tested by incubating the endolysin at temperatures ranging from 4° C. to 55° C. SP-CHAP displayed lytic activity on Spn up to 37° C. (FIG. 1B) with a dramatic decrease in activity at 40° C., 45° C., and 55° C., suggesting a melting temperature (Tm) between 37° C. and 40° C. This Tm is consistent with melting temperatures of other pneumococcal endolysins, such as 43.5° C. for Cpl-1 (12), 37° C. for Pal (13), and 43.9° C. for Cpl-7 (14). The antibacterial effectiveness of several endolysins has been shown to increase with the addition of NaCl (15). However, using a constant concentration of 25 μg/mL SP-CHAP, an inverse correlation between NaCl concentration and enzymatic activity was observed (FIG. 1C). These results suggest the importance of ionic interactions for the bacterial surface, a phenomenon that has also been noted in the Bacillus-specific endolysins PlyP56, PlyN74, and PlyTB40 (16).

SP-CHAP has Greater Lytic Activity than Cpl-1 and Activity can be Partially Inhibited by Pneumococcal Capsule

To benchmark SP-CHAP against Cpl-1, their bacteriolytic activity was measured against several pneumococcal serovars and oral or nasal commensal organisms. While both SP-CHAP and Cpl-1 killed all pneumococcal strains tested, SP-CHAP appeared to be more effective against three of the five strains (FIG. 1D). Of note, the two endolysins did not kill the commensal organisms tested (FIG. 1E). Next, we performed a colony-forming unit (CFU) assay to quantify the lytic potential of SP-CHAP. With a 1 h treatment, a dose-dependent effect of SP-CHAP lytic activity on Spn Lyt4.4 was observed at concentrations from 1.56 to 100 μg/mL (FIG. 1F). Treatment of Spn with 100 μg/mL SP-CHAP resulted in 7 log 10 decrease in Lyt4.4 CFU, while Cpl-1 caused only a 2-log reduction at the same concentration. However, there was not a significant difference in activity between SP-CHAP and Cpl-1 at 12.5-1.56 μg/mL. To further quantify SP-CHAP activity, we performed standard minimum inhibitory concentration (MIC) assays. Cpl-1 displayed higher antimicrobial activity with a lower MIC than SP-CHAP against several Spn strains tested (FIG. 1G). We attribute these observed disparities in the activity of Cpl-1 and SP-CHAP to differences in the assay methodologies, as previously documented for other cell wall hydrolases (17). Specifically, the turbidity reduction assay and the log-fold killing assays involved incubating the enzymes with bacteria for 30 min or 1 h, primarily assessing bacteriolytic activity. In contrast, the MIC assays require overnight incubation of enzymes with bacteria, primarily assessing bacteriostatic activity.

These results disclosed herein highlighted variations in SP-CHAP activity against the encapsulated D39 strain and its capsule-free derivatives, R6 and Lyt4.4. Consequently, it was hypothesized that the presence of capsular polysaccharide may modulate SP-CHAP activity. This hypothesis stems from previous findings demonstrating that capsule shedding is a known pneumococcal response to antimicrobial peptides (18) and that the presence of a capsule in Klebsiella pneumoniae is linked to reduced endolysin activity (19). Pre-incubation of the purified capsular polysaccharide with the endolysins 30 min prior to and throughout the duration of a lytic assay resulted in a partial yet significant reduction in the lytic activity of SP-CHAP, but this effect was not observed with Cpl-1 (FIG. 1H).

SP-CHAP has Greater Activity than Cpl-1 Against Biofilm Pneumococci

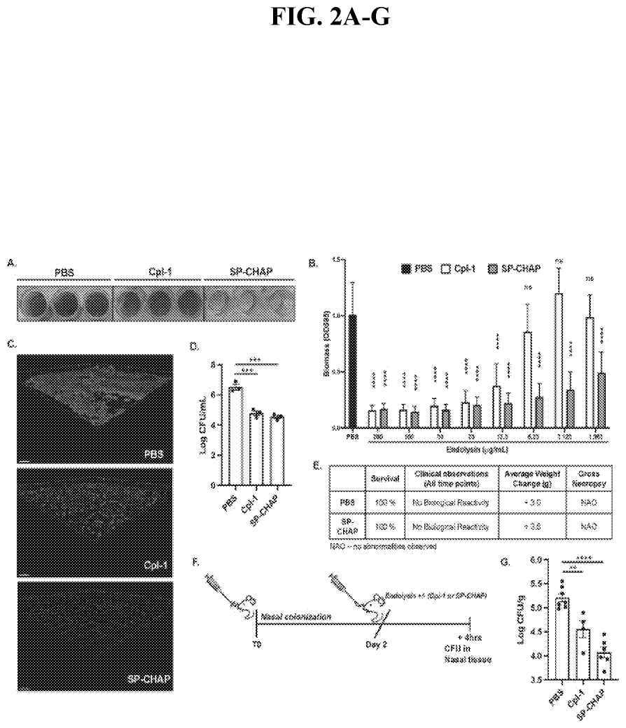

Spn can form biofilms to promote colonization of the nasopharynx and to improve antibiotic resistance (20). Using a static biofilm model, it was observed that SP-CHAP was highly efficient at eradicating pneumococcal biofilms when compared to Cpl-1 (FIG. 2A). Furthermore, when 24-h mature biofilms were treated with SP-CHAP for 1 h, it caused a dose-dependent decrease in biofilm volume at all concentrations tested, but Cpl-1 only dispersed biofilms at 12.5 μg/mL and higher concentrations (FIG. 2B). Supporting these observations, confocal imaging of biofilms demonstrated that biofilms treated with endolysins exhibited thinner structures compared to the control group, with SP-CHAP causing a greater reduction in biofilm thickness than Cpl-1 (FIG. 2C). These anti-biofilm properties were mirrored by similar decreases in Spn viability within the biofilms (FIG. 2D).

### SP-CHAP is not Toxic to the Host but Efficiently Decolonizes the Nasopharynx In Vivo

Using a model of toxicity, we evaluated the systemic response to SP-CHAP following intraperitoneal injection into mice. Using a total injection of 600 μg SP-CHAP or 10× the dose used in the decolonization studies below, we observed that none of the test animals exhibited any clinical signs of distress throughout the 3-day observation period. All animals from the test group gained weight, and no abnormalities were noted during gross necropsy (FIG. 2E). Finally, to assess the effectiveness of SP-CHAP as a decolonizing agent in vivo, we used a mouse Spn nasopharyngeal colonization model (FIG. 2F). We observed that intranasal administration of 60 μg SP-CHAP per mouse significantly reduced Spn CFUs in the nasal tissue, and this was slightly more pronounced than the effect of Cpl-1 (FIG. 2G). Of note, previous studies have demonstrated that intranasal delivery of 100 μg of penicillin was inadequate to decrease nasopharyngeal colonization by Spn (21), highlighting the advantages of endolysins over conventional antibiotics.

The results disclosed herein indicate that SP-CHAP is a non-host toxic, highly efficient endolysin with the ability to lyse Spn and decolonize biofilm-grown bacteria both in vitro and in vivo. Evidence that capsule can reduce the activity of SP-CHAP during planktonic growth also establishes a novel observation as a possible mechanism for pneumococci to protect themselves against this class of antimicrobial. Taken together, the data establishes the initial therapeutic capabilities of SP-CHAP and identify a potential mechanism employed by planktonic Spn to counter endolysin activity.

### REFERENCES

1. Danis-Wlodarczyk K M, Wozniak D J, Abedon S T. 2021. Treating Bacterial Infections with Bacteriophage-Based Enzybiotics: In Vitro, In Vivo and Clinical Application. Antibiotics (Basel) 10.
2. Linden S B, Alreja A B, Nelson D C. 2021. Application of bacteriophage-derived endolysins to combat streptococcal disease: current state and perspectives. Curr Opin Biotechnol 68:213-220.
3. Shenoy A T, Orihuela C J. 2016. Anatomical site-specific contributions of pneumococcal virulence determinants. Pneumonia (Nathan) 8.
4. Austrian R. 1981. Pneumococcus: the first one hundred years. Rev Infect Dis 3:183-9.
5. Jain S, Self W H, Wunderink R G, Team CES. 2015. Community-acquired pneumonia requiring hospitalization. N Engl J Med 373:2382.
6. CDC. 2019. Antibiotic resistance threats in the United States. U.S. Department of Health and Human Services, CDC, Atlanta, GA.
7. Loeffler J M, Djurkovic S, Fischetti V A. 2003. Phage lytic enzyme Cpl-1 as a novel antimicrobial for pneumococcal bacteremia. Infect Immun 71:6199-204.
8. Loeffler J M, Nelson D, Fischetti V A. 2001. Rapid killing of Streptococcus pneumoniae with a bacteriophage cell wall hydrolase. Science 294:2170-2.
9. Broendum S S, Buckle A M, McGowan S. 2018. Catalytic diversity and cell wall binding repeats in the phage-encoded endolysins. Mol Microbiol 110:879-896.
10. Alreja A B, Linden S B, Lee H R, Chao K L, Herzberg O, Nelson D C. 2023. Understanding the Molecular Basis for Homodimer Formation of the Pneumococcal Endolysin Cpl-1. ACS Infect Dis 9:1092-1104.
11. Domenech M, Garcia E, Moscoso M. 2011. In vitro destruction of Streptococcus pneumoniae biofilms with bacterial and phage peptidoglycan hydrolases. Antimicrob Agents Chemother 55:4144-8.
12. Sanz J M, Garcia J L, Laynez J, Usobiaga P, Menendez M. 1993. Thermal stability and cooperative domains of CPL1 lysozyme and its NH2- and COOH-terminal modules. Dependence on choline binding. J Biol Chem 268:6125-30.
13. Varea J, Monterroso B, Saiz J L, Lopez-Zumel C, Garcia J L, Laynez J, Garcia P, Menendez M. 2004. Structural and thermodynamic characterization of Pal, a phage natural chimeric lysin active against pneumococci. J Biol Chem 279:43697-707.
14. Bustamante N, Rico-Lastres P, Garcia E, Garcia P, Menendez M. 2012. Thermal stability of Cpl-7 endolysin from the Streptococcus pneumoniae bacteriophage Cp-7; cell wall-targeting of its CW_7 motifs. PLOS One 7: e46654.
15. Binte Muhammad Jai H S, Dam L C, Tay L S, Koh J J W, Loo H L, Kline K A, Goh B C. 2020. Engineered lysins with customized lytic activities against enterococci and staphylococci. Front Microbiol 11:574739.
16. Garcia P, Martinez B, Rodriguez L, Rodriguez A. 2010. Synergy between the phage endolysin LysH5 and nisin to kill Staphylococcus aureus in pasteurized milk. Int J Food Microbiol 141:151-5.
17. Kusuma C M, Kokai-Kun J F. 2005. Comparison of four methods for determining lysostaphin susceptibility of various strains of Staphylococcus aureus. Antimicrob Agents Chemother 49:3256-63.
18. Schmelcher M, Shen Y, Nelson D C, Eugster M R, Eichenseher F, Hanke D C, Loessner M J, Dong S, Pritchard D G, Lee J C, Becker S C, Foster-Frey J, Donovan D M. 2015. Evolutionarily distinct bacteriophage endolysins featuring conserved peptidoglycan cleavage sites protect mice from MRSA infection. J Antimicrob Chemother 70:1453-65.
19. Kietzman C C, Gao G, Mann B, Myers L, Tuomanen E I. 2016. Dynamic capsule restructuring by the main pneumococcal autolysin LytA in response to the epithelium. Nat Commun 7:10859.
20. Hong H W, Kim Y D, Jang J, Kim M S, Song M, Myung H. 2022. Combination effect of engineered endolysin EC340 with antibiotics. Front Microbiol 13:821936.
21. Blanchette-Cain K, Hinojosa C A, Akula Suresh Babu R, Lizcano A, Gonzalez-Juarbe N, Munoz-Almagro C, Sanchez C J, Bergman M A, Orihuela C J. 2013. Streptococcus pneumoniae biofilm formation is strain dependent, multifactorial, and associated with reduced invasiveness and immunoreactivity during colonization. mBio 4: e00745-13.
22. Marks L R, Clementi E A, Hakansson A P. 2012. The human milk protein-lipid complex HAMLET sensitizes bacterial pathogens to traditional antimicrobial agents. PLOS One 7: e43514.
23. Nelson D C, Schmelcher M, Rodriguez-Rubio L, Klumpp J, Pritchard D G, Dong S, Donovan D M. 2012. Endolysins as antimicrobials. Adv Virus Res 83:299-365.
24. Shenoy A T, Brissac T, Gilley R P, Kumar N, Wang Y, Gonzalez-Juarbe N, Hinkle W S, Daugherty S C, Shetty A C, Ott S, Tallon L J, Deshane J, Tettelin H, Orihuela C J. 2017. Streptococcus pneumoniae in the heart subvert the host response through biofilm-mediated resident macrophage killing. PLOS Pathog 13: e1006582.
25. Riegler A N, Brissac T, Gonzalez-Juarbe N, Orihuela C J. 2019. Necroptotic cell death promotes adaptive immunity against colonizing pneumococci. Front Immunol 10:615.

What is claimed is:

1. A pharmaceutical composition comprising:
(i) a SP-CHAP polypeptide, fragment or variant thereof; or
(ii) a nucleic acid that encodes for the SP-CHAP, fragment or variant thereof,
and a pharmaceutically acceptable carrier.

2. The pharmaceutical composition of claim 1, wherein the SP-CHAP protein is a polypeptide of SEQ ID NO:1, a polypeptide having at least about 90% or more homology with the SP-CHAP of SEQ ID NO:1 or a polypeptide fragment thereof that retains endolysin activity.

3. The pharmaceutical composition of claim 1, wherein the nucleic acid is codon optimized.

4. The pharmaceutical composition of claim 1, wherein the SP-CHAP contains a signal peptide.

5. The pharmaceutical composition of claim 1, wherein administration of the pharmaceutical composition results in a reduction in pneumococcal disease when administered to a subject having a pneumococcal infection.

6. The pharmaceutical composition of claim 5, further comprising a second therapeutic useful for treatment of the pneumococcal disease.

7. The pharmaceutical composition of claim 5, wherein the pneumococcal disease is selected from the group consisting of pneumonia, invasive pneumococcal disease, meningitis and sepsis.

8. The pharmaceutical composition of claim 1, wherein SP-CHAP-encoding nucleic acid is associated with a vector.

9. The pharmaceutical composition of claim 1, wherein the SP-CHAP-encoding nucleic acid is associated with a lipid-based composition.

10. The pharmaceutical composition of claim 1, formulated for intranasal administration.

11. The pharmaceutical composition of claim 1, wherein the SP-CHAP, or SP-CHAP encoding nucleic acid, is associated with a nanoparticle.

12. The pharmaceutical composition of claim 9, wherein the SP-CHAP, or SP-CHAP encoding nucleic acid is associated with a liposome.

13. A method for treating a pneumococcal disease in a subject infected with a pneumococcal bacterium, the method comprising administering to the subject, an effective amount of the pharmaceutical composition of claim 1.

14. The method of claim 13, wherein the pneumococcal disease is selected from the group consisting of pneumonia, invasive pneumococcal disease, meningitis and sepsis.

15. The method of claim 13, wherein the pharmaceutical composition of claim 1 is co-administered with a second therapeutic useful for treatment of the pneumococcal disease.

16. The method of claim 13, wherein the second therapeutic is an anti-bacterial agent.

17. The method of claim 13, wherein the pharmaceutical composition of claim 1 is administered intranasally.

18. A kit comprising the pharmaceutical composition or claim 1 and instructions for use in treating a pneumococcal disease.


SEQUENCE LISTING (2 sequences)

SEQ ID NO: 1 - protein, 288 aa, synthetic construct
MSKKQEMIQFFIDKANSGDGVDNDGAYGFQCADVPCYGLRHWYGVTLWGNAYDLLESARSQGLKVVYDAEYPKAGWFFVKSYVAGDGVNYGHTGLVYEDSDGSTIKTIEQNIDGNWDYLEVGGPCRYNERSVSEIVGYIVPPEEVEIGWQQNQYGWWWVREDGSYPTEKWEKINDVWYYFDDKGFMKRSTWLNYKDAWYWFTDSGSMAIGWARINNAWYYFDEEGKMVTGWIKHKLTWYYLDGKEGTMVSNAFIQSADKKGWYYLKPDGTMEDKPEFEIEPDGLITTK

SEQ ID NO: 2 - DNA, 864 bp, synthetic construct
  source 1..864: mol_type = other DNA
atgagcaaaaagcaagagatgatccagttcttcatcgataaagcaaatagcggtgatggcgttgataatgatggtgcatatggttttcagtgtgcagatgttccgtgttatggtctgcgtcattggtatggtgttaccctgtggggtaatgcatatgacctgctggaaagcgcacgtagccagggtctgaaagttgtttatgatgcagaatatccgaaagccggttggtttttcgttaaaagctatgttgccggtgatggtgtgaattatggtcataccggtctggtttatgaagatagtgatggtagcaccattaaaaccatcgaacagaatattgatggcaactgggattatctggaagttggtggtccgtgtcgttataatgaacgtagcgttagcgaaattgtgggctatattgttccgcctgaagaagttgaaattggttggcagcagaatcagtatggttggtggtgggttcgtgaagatggtagctatccgaccgaaaaatgggagaaaattaacgatgtgtggtactacttcgatgacaagggttttatgaaacgtagcacctggctgaactataaagatgcatggtattggtttaccgatagcggtagcatggccattggttgggcacgtattaacaatgcctggtattatttcgacgaagagggtaaaatggttaccggctggattaaacataaactgacgtggtattacctggatggtaaagaaggcaccatggttagcaatgcatttattcagagcgcagataagaaaggctggtactatctgaaaccggatggtacaatggaagataaaccggaatttgaaattgagccggatggtctgattaccaccaaa